(19) United States
(12) Patent Application Publication
(10) Pub. No.: US 2013/0144262 A1
(43) Pub. Date: Jun. 6, 2013

(54) Package for Delivering Microdoses of Medicament

(71) Applicants: Bernd Kuhn, Frankfurt am Main (DE); Daniel Wagner, Frankfurt am Main (DE)

(72) Inventors: Bernd Kuhn, Frankfurt am Main (DE); Daniel Wagner, Frankfurt am Main (DE)

(73) Assignee: SANOFI-AVENTIS DEUTSCHLAND GMBH, Frankfurt am Main (DE)

(21) Appl. No.: 13/816,455

(22) PCT Filed: Aug. 24, 2011
(86) PCT No.: PCT/EP2011/064505

§ 371 Date: Feb. 11, 2013

Related U.S. Application Data
(60) Provisional application No. 61/377,580, filed on Aug. 27, 2010.

(30) Foreign Application Priority Data
Oct. 8, 2010 (EP) 10186926.1

(51) Int. Cl. A61J 1/14; A61M 5/19

(52) U.S. Cl. CPC A61J 1/1412; A61J 1/1406; A61M 5/19
USPC 604/506; 604/403; 604/414

(57) ABSTRACT

A primary package for simultaneously dispensing microdoses of from 5 to 500 microliters of one medicament contained in a medicated module and another medicament contained in an attached injection system is disclosed, where the primary package contains a single dose of medicament contained within a reservoir cavity sealed with closure caps that secure top and bottom septa.

### CROSS REFERENCE TO RELATED APPLICATIONS

The present application is a U.S. National Phase Application pursuant to 35 U.S.C. §371 of International Application No. PCT/EP2011/064505 filed Aug. 24, 2011, which claims priority to U.S. Patent Application No. 61/377,580 filed Aug. 27, 2010 and European Patent Application No. 10186926.1 filed Oct. 8, 2010. The entire disclosure contents of these applications are herewith incorporated by reference into the present application.

### FIELD OF INVENTION

Specific embodiments of the present disclosure relate to microdose size medicament packages for use with medical devices and methods of delivering at least two drug agents from separate reservoirs using devices having only a single dose setting mechanism and a single dispense interface. A single delivery procedure initiated by the user causes a non-user settable microdose of a secondary drug agent and a variable set dose of a primary drug agent to be delivered to the patient. More specifically, the present disclosure relates to a low part count package design for a microdose of the secondary medicament suitable for a high-speed medicament line filling process.

### BACKGROUND

Certain disease states require treatment using one or more different medicaments. Some drug compounds need to be delivered in a specific relationship with each other in order to deliver the optimum therapeutic dose. The present disclosure is of particular benefit where combination therapy is desirable, but not possible in a single formulation for reasons such as, but not limited to, stability, compromised therapeutic performance and toxicology.

For example, in some cases it might be beneficial to treat a diabetic with a long-acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus.

There are a number of potential problems when delivering two active medicaments or “agents” simultaneously. The two active agents may interact with each other during the long-term, shelf life storage of the formulation. Therefore, it is advantageous to store the active components separately and only combine them at the point of delivery, e.g. injection, needle-less injection, pumps, or inhalation. However, the process for combining the two agents needs to be simple and convenient for the user to perform reliably, repeatedly and safely.

A further problem is that the quantities and/or proportions of each active agent making up the combination therapy may need to be varied for each user or at different stages of their therapy. For example, one or more actives may require a titration period to gradually introduce a patient up to a “maintenance” dose. A further example would be if one active requires a non-adjustable fixed dose while the other is varied in response to a patient's symptoms or physical condition. This problem means that pre-mixed formulations of multiple active agents may not be suitable as these pre-mixed formulations would have a fixed ratio of the active components, which could not be varied by the healthcare professional or patient or user.

Additional problems may arise where a multi-drug compound therapy is required, because many users cannot cope with having to use more than one drug delivery system or make the necessary accurate calculation of the required dose combination. This is especially true for users with dexterity or cognitive difficulties. Because the fixed-dose or secondary medicament is a microdose in the range of from about 5 to about 500 microliters, the overall size of package containing the microdose is very small and the parts that make up the package are even smaller.

Accordingly, there exists a strong need to provide a package design that uses inert materials that will not interact with the secondary medicament, that complies with known regulatory requirements, and that is large enough to permit exact filling in an automatic line filling process. The present disclosure overcomes the above-mentioned problems by providing a package containing a molded reservoir that holds the microdose and that has a low part count that is easily assembled in a high-speed automated manufacturing process. The use of in-molding of functional features or of component parts greatly reduces the part count and snap fit closures to seal the reservoir cavity or inserted tube and allow for efficient aseptic manufacturing of the package. These and other advantages will become evident from the following more detailed description of the invention.

The problem to be solved by the present invention is to provide a package, a medicated module and a method where the safety of the user is increased.

### SUMMARY

The present disclosure relates to a microdose size package of medicament, preferably for use in a medicated module that allows complex combinations of multiple drug compounds within a single drug delivery system. With the microdose package attached to an injection device the user can set and dispense a multi-drug compound device through one single dose setting mechanism and a single dispense interface. This single dose setter may control the mechanism of the device such that a predefined combination of the individual drug compounds is delivered when a single dose of one of the medicaments is set and dispensed through the single dispense interface.

Defining the therapeutic relationship between the individual drug compounds in the drug delivery system would help to ensure that a patient/user receives the optimum therapeutic combination dose from a multi-drug compound device without the inherent risks associated with multiple inputs where the user has to calculate and set the correct dose combination every time they use the device. The microdose medicament in the package of the present disclosure is preferably a single dose of a liquid, suspension or emulsion.

In a preferred embodiment, a master drug compound, such as insulin, contained within a multiple dose, user selectable injection device could be used with a single use, user replaceable, module that contains a single microdose of a secondary medicament and a single dispense interface. When connected to the primary device, the secondary drug compound may be delivered on dispense of the primary compound. Although the present disclosure specifically mentions insulin, insulin analogs or insulin derivatives, and GLP-1 or GLP-1 analogs as two possible drug combinations, other drugs or drug combinations, such as an analgesics, hormones, beta agonists or corticosteroids, or a combination of any of the above-mentioned drugs could be used with our invention.

For the purposes of our invention the term “insulin” shall mean Insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

As used herein the term “GLP-1” shall mean GLP-1, GLP-1 analogs, or mixtures thereof, including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2), Exendin-3, Liraglutide, or AVE0010 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH2).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

In particular, the present disclosure is aimed at efficiently expelling or flushing the microdose of secondary medicament from the package contained in a medicated module such that at least about 80%, preferably greater than 90%, is expelled from the device through the single dispense interface. In some instances there may be a minimum dose of the first medicament that must be delivered to a patient. In those situations, it is important that the “flush through” performance of the system being as high as possible to expel substantially all of the second medicament when the lowest dose of the first medicament is set by the user. Initial studies show that by minimizing the volume of the reservoir, the theoretical “flush through” performance increases. For example, decreasing the volume of the reservoir from 40 microliters to 25 microliters and dispensing a two unit dose of the first medicament equivalent to 20 microliters will increase the flush through of the second medicament from about 50% to about 80%.

Preferably, the secondary medicament is expelled with a minimum of mixing with the primary medicament. This can be accomplished by designing the medicated module with a reservoir having an integral flow distribution device or a collection of separate parts defining a flow distribution system. The terms “flow distributor,” “flow distribution device,” and “flow distribution system” are used interchangeably in this application and are meant to include all flow configurations that increase the expulsion of the second medicament from the medicated module, preferably to a level of at least about 80%. In some cases the flow distribution system may be a single component and in other embodiments it may be a collection of components, such as ribs, spirals, grooves, or channels that are defined using various structures in the medicated module. Likewise, all or part of the flow distribution system may be in-molded during fabrication of the reservoir using the same or different construction materials. Alternatively, the flow distribution system may be a separate part or collection of parts that are permanently fixed in the reservoir with glue, welds, chemical bonds or any like fixation methods.

According to one aspect, a primary package is provided. The primary package may be for use in a medicated module. The package may comprise a reservoir. The reservoir may contain a medicament, preferably one single dose of the medicament. The single dose may be a microdose of medicament. The reservoir may have a wall. The wall may have an outer surface, i.e. an outer wall, and an inner surface. The reservoir may have a proximal end. The reservoir may have a distal end. The reservoir may have a reservoir cavity. The reservoir may have at least one of preferably a plurality of connectors. The connectors may be in the outer wall. The connectors may be located at the proximal and/or distal ends of the reservoir. The package may comprise a top closure. The connectors may engage the top closure. In particular, the top closure may be engaged with the connector located at the proximal end of the reservoir. The package may comprise a bottom closure. The connectors may engage the bottom closure. In particular, the bottom closure may be engaged with the connector located at the distal end of the reservoir. The package may comprise a top seal. The top seal may be fitted between the top closure and the proximal end of the reservoir. The top seal may comprise a sealing disc. The package may comprise a bottom seal. The bottom seal may be fitted between the bottom closure and the distal end of the reservoir. The bottom seal may comprise a sealing disc. The top closure and/or the bottom closure may securely hold the respective sealing disc or septum at the distal and/or proximal ends of the reservoir. The connectors may be any attachment means that will allow the closures to be securely attached to the reservoir such that a sealing disc is maintained in a liquid tight seal with the reservoir. A preferred connector would be the female portion of a snap fit.

The top seal may be fitted between the top closure and the proximal end of the reservoir. Preferably, fitting between would mean that the top closure and the top seal are at least partially in contact. The fit may be form fit, positive fit, force closure, closed linkage, bonding, or any combination thereof. The top seal may comprise a sealing disc. The package may comprise a bottom seal. The bottom seal may be fitted between the bottom closure and the distal end of the reservoir. Preferably, fitting between would mean that the bottom closure and the bottom seal are at least partially in contact. The fit may be form fit, positive fit, force closure, closed linkage, bonding, or any combination thereof. The bottom seal may comprise a sealing disc. The top closure and/or the bottom closure may securely hold the respective sealing disc or septum at the distal and/or proximal ends of the reservoir. Holding the sealing disc or septum may securely position the sealing disc or septum to ensure proper positioning. Preferably, fixing the top/bottom seal between the top/bottom closure would fix the top/bottom seal in its position and tightly or sealingly close the reservoir. Further, the function of providing a liquid tight seal is ensured by properly positioning the sealing disc or septum. Preferably, the fit would hold the respective sealing disc or septum at the respective end of the reservoir to ensure that a sealing disc or septum is maintained in a liquid tight seal with the reservoir.

The sealing discs may be induction seals, septa, other pierceable seals or membranes comprised of rubber, polymer, or any other material that allow piercing by a hollow needle and that can be used to achieve an appropriate seal with the reservoir and closures. The volume of the reservoir cavity may be between about 5 microliters and about 500 microliters. The connectors and closures may be connected by a snap-lock engagement. In particular, the closures may engage the connectors in the outer wall through a snap ring or lock design, preferably one where the closures contain a rib or bead around the circumference that snaps into a similarly sized ring or groove around the top and/or bottom circumference of the reservoir.

The connectors and closures may be connected by a snap-lock engagement to the reservoir. The connectors and closures may remain connected to the reservoir once engaged. The closures may be attached to the reservoir while the seals are broken. The closures may be configured to allow the seals to be broken. Preferably, the connectors and closures may not need to be removed from the reservoir to pierce the seals. Preferably, the sealing disc or septum may be pierced while the closure is connected to the reservoir. In particular, the top seal may be fitted between the top closure and the proximal end of the reservoir when pierced by a needle. In particular, the bottom seal may be fitted between the bottom closure and the distal end of the reservoir when pierced by a needle. The top and/or bottom closure may comprise an opening, aperture, or cut-out configured to allow breaking the seal without affecting the closure. Preferably, breaking the seal does not affect the closure. Prefeably, breaking the seal does not affect that the seal is fitted between the closure and the end of the reservoir. The seals may be broken by way of a needle or cannula piercing the seal. Preferably, the seals are pierced without affecting the closure. Preferably, piercing the seals does not affect that the seal is fitted between the closure and the end of the reservoir.

According to an embodiment, the reservoir, the closures, the connectors and/or the seals are fabricated from materials selected from the group consisting of medical grade of plastic , silicon oxide coated plastic, glass, silicon, thermoplastic elastomers, rubber elastomers and a combination thereof.

Components of the reservoir can be made of plastic or glass. In the latter case, glass of hydrolytic type I or II as defined by either the United States or European pharmacopeia can be used. Examples of plastics that can be used are those that are medical grade and that are suitable as primary packaging for pharmaceutical preparations. These include for example polyethylene, polypropylene, cycloolefin copolymers, polybutylenterephthalat, cellulose acetate, polystyrene or polycarbonate. Plastics, that are suitable for closure elements and for flow distribution systemes are elastomers such as silicons, thermoplastic elastomers, or rubber elastomers suitable as primary packaging for pharmaceutical preparations. Other examples include TPE (thermo plastic elastomer), LSR (liquid silicone rubber), LDPE (low density polyethylene), and/or any kind of medical grade rubber, natural or synthetic. A transparent design of the reservoir is preferred as it may allow a visual check of the reservoir. However, opaque or only partially transparent forms are also conceivable.

According to an embodiment, the cavity contains a microdose, preferably a single microdose, of a medicament. The microdose may be a liquid medicament. Alternatively, the microdose may comprise a powdery medicament. The medicament may comprise a GLP-1 and/or insulin.

The secondary medicament may be contained directly in the reservoir, however, one preferred approach is to use a tube. The tube may be inserted into the reservoir. The tube is preferably one made of plastic or inert glass. The reservoir may also contain an injection molded membrane at the distal end of the reservoir. Thus, the side walls and distal end wall can be formed using the same plastic material, e.g. polyethylene, being in contact with the drug product and so minimizing the number of materials that need to be compatible with the drug product. As such plastic material typically does not have the necessary sealing properties to give a tight seal against a piercing needle, an additional elastomeric septum at the distal end may be required. The distal sealing disc or septum can be fitted against this membrane and the bottom closure secured to the reservoir wall prior to filling with the microdose of medicament. Alternatively, the distal sealing disc may be in-molded to the reservoir prior to filling with medicament. The distal sealing disc may also be in-molded into the reservoir by a multi-component injection molding process. This use of in-molding the lower (distal) sealing disc can be made with or without the membrane at the bottom of the reservoir.

To use the properties of glass regarding permeation barrier and drug product compatibility and the functionality of plastic materials allowing multiple shapes, combination of glass and plastic materials to form a reservoir is possible. For such a reservoir, a glass tube could be inserted into a plastic tube or could be over-molded by a plastic material. To achieve the properties of glass on a plastic surface, plastic tubes could also be coated with thin layers of silicon oxides. Such a coating is possible for example on polycarbonate plastic materials.

According to an embodiment, the cavity contains a flow distribution system. The flow distribution system may be in-molded in or permanently fixed to the reservoir cavity. The flow distribution system may be attached to at least one of the upper closure, the lower closure, and at least one component of the inner surface of the wall. The flow distribution system may comprise an insert. The insert may be placed concentrically within the cavity. The insert may comprise two or more ribs or grooves. The ribs or grooves may define one or more medicament flow channels. The medicament may be contained in the flow channels. In particular, the medicament may fill the flow channels. The insert may cause radial flow of medicament. The insert may have proximal and distal needle cavities.

According to an embodiment, the flow distribution system comprises one or more radial vanes.

In order to prevent back mixing of the microdose of medicament with the primary medicament during injection and to ensure plug flow of the microdose, preferably the flow distribution system is placed in the drug reservoir cavity. Such a system may minimize stagnant flow of the medicament and may promote/maximize plug flow of the medicament through the reservoir and out of the medicated module. The flow distribution system preferably contains the previously mentioned insert. The insert can be any structure or collection of structures/features that may cause radial and/or annular and/or axial and/or circumferential/swirl flow of the medicament and that significantly reduces or prohibits back mixing from occurring, and/or stagnant zones from forming. All or parts of the flow distribution system may be in-molded as part of the manufacture of the reservoir. Preferably, the flow distributor is a cylindrical pin or insert having two or more support ribs or flow grooves defining one or more medicament flow channels. Preferably, the flow distributor comprises one or more radial vanes. The use of the flow distributor may also greatly improve the efficiency related to the filling of the reservoir during manufacture of the reservoir and/or medicated module generally. Also preferred is to manufacture the insert with needle cavities so that upon activation of the reservoir during dose injection the distal and proximal needles in the medicated module are not impeded or damaged by the insert.

The size of the flow distribution system may be chosen such that the cross-sectional area of an annulus formed between the flow distribution system and the wall of the cavity is minimized. In particular, an outer diameter of the flow distribution system may be only marginally smaller than the diameter of the cavity. Accordingly, only a small volume within the cavity may be left for storing the medicament in the cavity. Accordingly, only a single microdose of medicament may be stored in the cavity. The size of the microdose may depend on the size of the flow distribution system and the size of the inner cavity.

The size of the reservoir and the flow distribution system may be chosen such that the package comprises a size which is suited for handling, transport, manufacture and assembly. In particular, the reservoir and the flow distribution system and, thus, the package may comprise a size which is large enough to be handled during an automatic manufacturing procedure.

The size of the reservoir of the present disclosure may be in the range of from about 5 to about 500 microliters. In one embodiment the volume available to store the medicament would equal the internal volume of the reservoir minus the volume of the flow distributor or flow distribution system. Therefore, if the volume of the flow distributor is marginally smaller than the internal volume of the reservoir, a small volume is left which the medicament occupies. Hence, the scale of both the reservoir and the flow distributor can be large while storing a small volume of medicament. As such, the external package or reservoir geometry is not dictated by the volume of medicament. As a result, for small volumes of medicament (e.g. 5-500 microliters) the package can be of an acceptable size for handling, transport, manufacture and assembly.

The reservoir is preferably sterilized before filling with the microdose of medicament and then sealed after filling to maintain sterility. Preferably, the reservoir contains a liquid medicament, most preferably comprising a glucagon-like peptide-1 (GLP-1). In some cases, it is preferred that the reservoir contains a liquid medicament comprising a mix of at least two drug agents, for example, a mix of a glucagon-like peptide-1 (GLP-1) and insulin.

According to an embodiment, the package comprises a gasket. The gasket may be located on the proximal end of the reservoir. The gasket may be in contact with at least one of the top closure, the distal end of the reservoir or the bottom closure.

According to an embodiment, the reservoir has a bypass channel. The bypass channel may connect the proximal end to the distal end. The bypass channel may not be in fluid communication with the cavity.

Another embodiment covers a medicated module where a first needle is fixed within a retention cap positioned in the proximal end of the module housing. A second needle may be fixed within the distal end of the housing. The previously described package comprising the reservoir having top and bottom seals or septa may be configured for fluid engagement with the first and second needle. The package may contain a single microdose of a secondary medicament. The medicated module may have retention features engaging the reservoir, such as spring washer or the like. In a more preferred embodiment, the first and second needles pierce the top and bottom seals, respectively, when the medicated module is attached to a drug delivery device. The retention cap can be configured to move axially in the distal direction when the medicated module is attached to the drug delivery device. In some cases where priming of the drug delivery system is desirable, the package within the medicated module may have a bypass to allow medicament from the primary medicament cartridge to flow around the reservoir in the package and exit the second needle. The bypass can be any configuration, such as a channel, pipe, conduit, groove, slot, or any other like pathway that is capable of carrying the medicament from the primary reservoir to the second needle without communicating with the secondary reservoir/medicament. Such a bypass may allow the multi use injection device to be primed and also both the primary and secondary needles to be primed without expelling any of the volume of the secondary medicament. Alternatively, the bypass channel may be used to inject only the primary medicament.

A further aspect relates to a method for dispensing a fixed microdose of a secondary medicament from a reservoir. The medicament may be dispensed for test purposes. The reservoir may be suited to minimize a volume in an inner reservoir cavity. In a first step, the previously described medicated module may be provided. The medicated module may comprise the primary package holding the single microdose of the secondary medicament. In a next step, the medicated module may be permanently or releasably attached to a drug delivery device. The drug delivery device may comprise a cartridge. The cartridge may hold a primary medicament, preferably a plurality of doses of the primary medicament. In a next step, a dose of the primary medicament may be set. In a next step, the set dose of the primary medicament may be moved from the cartridge in a distal direction. The primary medicament may be moved such that the microdose of the secondary medicament, in particular the complete microdose, is moved from the reservoir. The flow distribution system of the primary package may be configured to prevent mixing of the secondary medicament with the primary medicament when the primary medicament is moved in the distal direction.

The present disclosure also covers the method of dispensing a fixed dose of one medicament and a variable dose of a primary medicament from separate reservoirs that involves the steps of first setting a dose of a first medicament contained in a primary reservoir a drug delivery device having a single dose setter. Next a dose button is activated that moves the set dose of the first medicament from the primary medicament cartridge in a distal direction and simultaneously forces substantially all of a non-user settable dose (e.g. a single microdose) of a second medicament from a sealed reservoir having an integral flow distributor, where the reservoir is contained in a removable/disposable medicated module through a single dispense interface, preferably a hollow injection needle. Upon completion of the delivery procedure, substantially all of the second medicament has been expelled as well as the set dose of the first medicament through the single dispense interface. By “substantially all” we mean that at least about 80% of the second medicament is expelled from the drug delivery device, preferably at least about 90% is expelled from the reservoir. In one arrangement, preferably at least about 80% is delivered within the first 50 microliters (5 units) flow of the primary medicament.

A particular benefit of the present disclosure is that the reservoir in the medicated module makes it is possible to tailor dose regimes when required, especially where a titration period is necessary for a particular drug. The medicated module could be supplied in a number of titration levels with obvious differentiation features such as, but not limited to aesthetic design of features or graphics, numbering etc, so that a patient could be instructed to use the supplied medicated module in a specific order to facilitate titration. Alternatively, the prescribing physician may provide the patient with a number of “level one” titration medicated modules and then when these were finished, the physician could then prescribe the next level. A key advantage of this titration program is that the primary injection device remains constant throughout.

A further aspect relates to a flow distribution system. The flow distribution system may be configured for reducing a volume of a medicament reservoir, e.g. the previously mentioned medicament reservoir. The reservoir may hold a medicament, preferably one microdose of the medicament. The flow distribution system may comprise a main body. The main body may comprise the previously described insert. The main body may comprise a pin. The main body may be configured to ensure that the medicament is arranged on an outer part of the volume of the medicament reservoir, e.g. between the wall of the reservoir and the main body of the flow distribution system. The flow distribution system may comprise at least one rib or groove. The at least one rib or groove may define one or more medicament flow channels. The medicament may fill the one or more flow channels.

According to a preferred embodiment, a primary package for use in a medicated module is provided. The primary package comprises a reservoir having a proximal end, a distal end and a reservoir cavity, wherein the reservoir is configured to contain a medicament. The primary package comprises connectors located at the proximal and distal ends of the reservoir. The primary package comprises a top closure engaged with the connector located at the proximal end of the reservoir. The primary package comprises a bottom closure engaged with the connector located at the distal end of the reservoir. The primary package comprises a top seal fitted between the top closure and the proximal end of the reservoir. The primary package comprises a bottom seal fitted between the bottom closure and the distal end of the reservoir. The connectors are configured to allow the closures to be securely attached to the reservoir such that the respective seal is maintained in a liquid tight seal with the reservoir.

According to a preferred embodiment, a medicated module attachable to a drug delivery device is provided, the drug delivery device comprising a cartridge of a primary medicament and the medicated module comprising a first needle, a second needle and the previously described primary package. The primary package is configured for fluid engagement with the first and second needles.

According to a preferred embodiment, a primary package for use in a medicated module is provided, the primary package comprising a reservoir having a wall comprising an outer surface and an inner surface, a proximal end, a distal end and a reservoir cavity. The primary package comprises connectors in the outer surface of the wall located at the proximal and distal ends of the reservoir. The primary package comprises a top closure engaged with the connector located at the proximal end of the reservoir. The primary package comprises a bottom closure engaged with the connector located at the distal end of the reservoir. The primary package comprises a top seal fitted between the top closure and the proximal end of the reservoir. The primary package comprises a bottom seal fitted between the bottom closure and the distal end of the reservoir.

According to a preferred embodiment, a method for dispensing a fixed microdose of a secondary medicament from a reservoir which is suited to minimize a volume in an inner reservoir cavity is provided. The method comprises the step of providing the previously described medicated module. The method comprises the step of attaching the medicated module to a drug delivery device, the drug delivery device comprising a cartridge holding a primary medicament. The method comprises the steps of setting a dose of the primary medicament and moving the set dose of the primary medicament from the cartridge in a distal direction such that the microdose of the secondary medicament is moved from the reservoir.

According to a preferred embodiment, a flow distribution system configured for reducing a volume of a medicament reservoir holding a medicament is provided. The flow distribution system comprises a main body configured to ensure that the medicament is arranged on an outer part of the volume of the medicament reservoir. The flow distribution system comprises at least one rib or at least one groove defining one or more medicament flow channels, wherein the medicament fills the one or more flow channels.

These as well as other advantages of various aspects of the present invention will become apparent to those of ordinary skill in the art by reading the following detailed description, with appropriate reference to the accompanying drawings.

The scope of the invention is defined by the content of the claims. The invention is not limited to specific embodiments but comprises any combination of elements of different embodiments. Moreover, the invention comprises any combination of claims and any combination of features disclosed by the claims.

### BRIEF DESCRIPTION OF THE DRAWINGS

Exemplary embodiments are described herein with reference to the drawings, in which:

FIG. 1 illustrates one possible drug delivery device;

FIG. 2 illustrates an embodiment of the package having a self contained reservoir capable of holding a microdose of medicament having two pierceable sealing discs;

FIG. 3 illustrates an embodiment of the package having a tube inserted in the cavity of the reservoir and having the distal closure connected to the outer wall of the reservoir and having the top (proximal) end open for filling of the microdose of medicament;

FIG. 4 illustrates an embodiment of the package where the reservoir cavity contains a flow distribution system having needle cavities and being attached to the proximal closure element;

FIG. 5 illustrates a medicated module containing an embodiment of the package having a flow distribution system contained with the reservoir cavity;

FIG. 6 illustrates a close-up of one embodiment of an annular flow distributor contained in the reservoir;

FIG. 7 illustrates medicament flow paths in using one embodiment of the flow distributor contained in the reservoir;

FIG. 8 illustrates a close-up of another embodiment of the flow distributor having radial vanes contained in the reservoir;

FIG. 9 illustrates theoretical medicament flow paths using the annular flow distributor illustrated in FIG. 8;

FIG. 10 illustrates a close-up of number of other possible embodiments of the flow distributor;

FIG. 11 illustrates an embodiment of the package where the reservoir cavity contains a flow distribution system being in-molded with the plastic wall of the reservoir;

FIG. 12 illustrates a cross-sectional view of a possible embodiment of the flow distributor; and

FIG. 13 illustrates an embodiment of the package where the outer wall of the reservoir cavity contains a bypass channel being in-molded.

### DETAILED DESCRIPTION

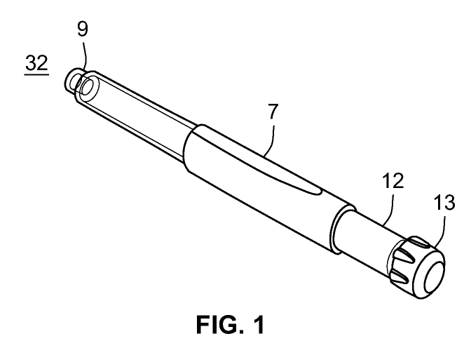

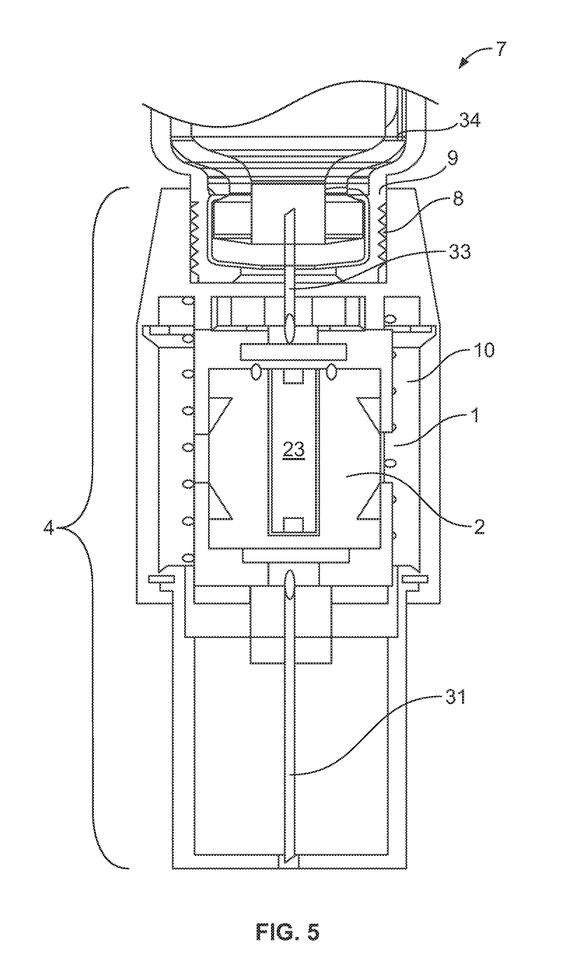

The present disclosure allows a fixed predetermined microdose of a secondary drug compound (medicament) to be administered to a patient simultaneously with a variable dose of a first primary or first drug compound through a single output or drug dispense interface. In a preferred embodiment, the drug dispense interface is a needle cannula (hollow needle). FIG. 1 illustrates one example of a drug delivery device 7 that a medicated module 4 (see FIG. 5) containing a package 1 can be attached. Each medicated module 4 is preferably self-contained and provided as a sealed and sterile disposable module that has an attachment means 8 compatible to an attachment means at the distal end 32 of device 7. Although not shown, the medicated module 4 could be supplied by a manufacturer contained in a protective and sterile container where the user would peel or rip open a seal or the container itself to gain access to the sterile medicated module 4. In some instances it might be desirable to provide two or more seals for each end of the medicated module 4.

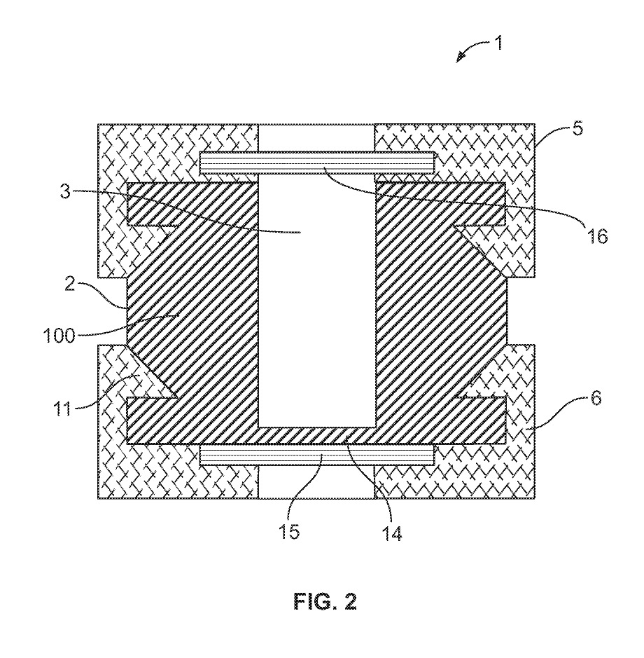

Any known attachment means 8 can be used to attach the medicated module 4 to the chosen drug delivery device 7, including all types of permanent and removable connection means, such as threads, snap locks, snap fits, luer locks, bayonet, snap rings, keyed slots, and combinations of such connections. FIG. 5 illustrates the attachment means 8 as screw threads that would engage threads 9 of the distal end 32 of drug delivery device 7. The embodiments shown in FIGS. 2-4 have the benefit of the microdose of the secondary medicament as a single dose being contained entirely within a reservoir 2, specifically in reservoir cavity 3, hence minimizing the risk of material incompatibility between the medicament and the materials used in the construction of the medicated module 4, specifically of a housing 10 (see FIG. 5). This also permits the package 1 to be manufactured and assembled as a separate independent sub assembly. The use of the flow distribution system, such as the cylindrical insert 23 also significantly assists the filling of the reservoir.

FIG. 2 illustrates one embodiment showing package 1 which comprises reservoir 2 having an outer wall 100, the cavity 3, a top closure 5 (proximal end) and a bottom closure 6 (distal end) engaged with connectors 11. The distal end of the reservoir cavity 3 has an injection molded membrane 14 to seal the distal end with the same material as used for the outer wall 100. Adjacent to membrane 14 is sealing disc 15 that permits one of injection needles 31 (see FIG. 5) of the medicated module 4 to pierce it during dose delivery. On the proximal end of package 1 is the top closure 5 that holds a sealing disc 16 in place sealing the top of the cavity 3.

FIG. 3 illustrates another embodiment where the cavity 3 contains a tube 35, preferably made of glass, to prevent the microdose of medicament from contacting the reservoir cavity wall. The tube 35 forms a barrier against permeation through the wall. This design also illustrates that sealing disc 15 can be in-molded. This means that the sealing disc 15 is molded during the injection molding process of the reservoir wall, preferably in a multi-component molding process from plastic material (e.g. plastic elastomer). Alternatively, a pre-formed sealing disc 15 can be introduced together with the glass tube into a co-molding process and the outer reservoir walls are over-molded using plastic material. As a further alternative, the glass part or tube 35 could be formed by a coating process as a thin layer of silicon oxide coating known by the art on the plastic surface of the reservoir wall. Once the tube 15 is filled with medicament a top closure similar to top closure 5 and a sealing disc 15 are attached to the top of reservoir 2.

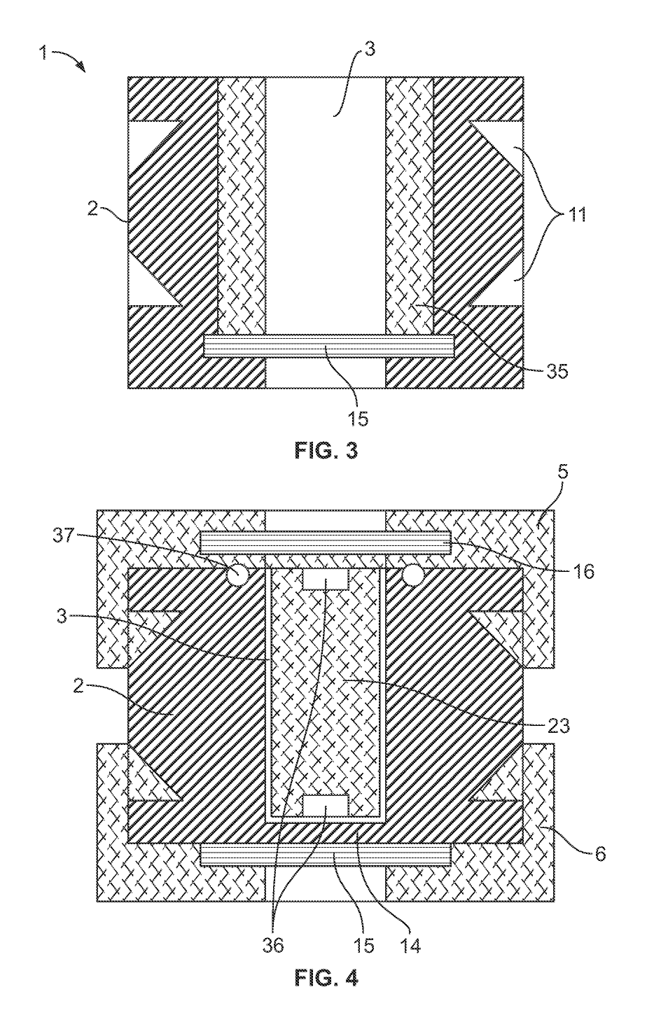

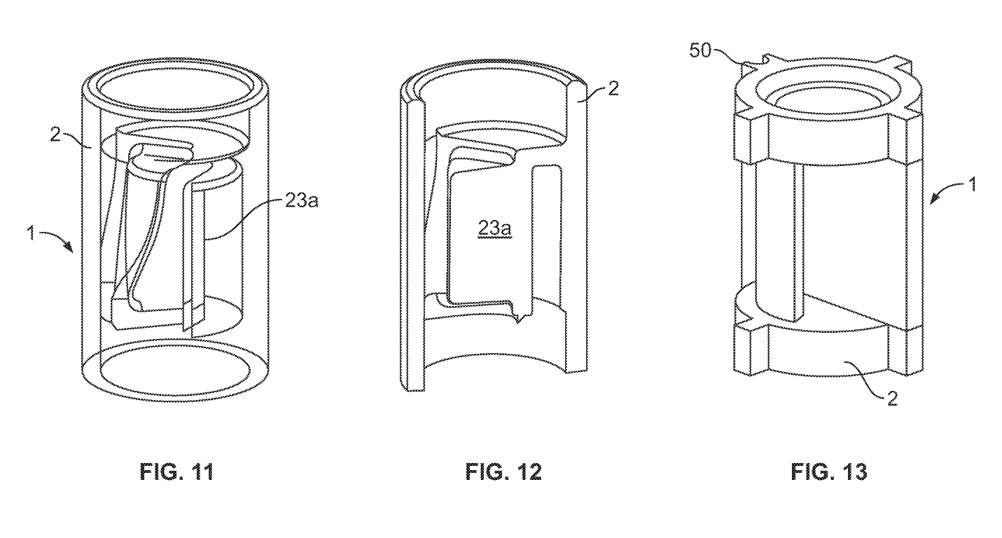

To minimize the residual volume of the microdose of the secondary medicament that is caused by recirculation and/or stagnant zones and that might remain in reservoir 2 at the end of the injection operation, a flow distributor 23 is located within cavity 3 as illustrated in FIG. 4. Preferably, closures 5 and 6 are made from an elastic material that can form a snap fit or snap lock with the connectors 11 on the reservoir outer wall 100. Alternatively, a crimpable material can be used to fabricate the closures 5, 6, most preferably a thin metal, such as aluminium. As further alternative, an outer snap-fit connector can be avoided as closure aid if the elastic septum can be inserted with a sufficient radial pressure fit into the tube 35 (compare bung positions in FIG. 12). FIG. 4 also shows that the flow distributor 23 can have top and bottom needle cavities 36. A sealing gasket 37 that seals cavity 3 from top or upper closure 5 and reservoir wall 2 is preferably in-molded in the upper closure 5 of the reservoir wall. The gasket 37 assures a tight seal of the cavity 3 as the materials of construction of the reservoir cavity 3 and upper closure 5 may not have sufficient tight sealing properties if pressed together. The flow distributor 23 preferably is made from the same material that constructs the upper closure 5. Flow distributor 23 is co-molded on the upper closure 5 to be inserted during the closing process of attaching the upper closure 5 after the reservoir cavity 3 was filled with the drug product. Thus, the number of parts to be handled and introduced in the aseptic manufacturing area can be minimized and the automated filling and assembly process can be facilitated. This embodiment is preferred if the inner wall of the reservoir cavity 3 is made of glass.

Preferably, the design of a flow distribution system, including flow distributor 23, should ensure that at least about 80% of the microdose of medicament is expelled from the reservoir 2 through the distal end of needle 31. Most preferably, at least about 90% should be expelled. Ideally, displacement of the primary medicament from the injection device 7 through the reservoir 2 into the proximal end of needle 31 will displace substantially all of the secondary medicament without substantial mixing of the two medicaments.

The flow distribution system contained in package 1 is used to minimize the risk of mixing occurring between the two medicaments during dispense, therefore promoting plug flow. In promoting plug flow it is desirable to minimize, or preferably prevent, change in the cross-sectional area perpendicular to the flow direction where the two medicaments come into contact with each other. While desirable to minimize, or preferably to prevent, change in cross-sectional area of the flow channel, the effect of this in a standard needle arrangement would be to increase the length of the flow channel for a fixed volume of the second medicament. This can result in an excessive and unacceptable axial length of the medicated module. Using the flow distribution system provides a fluid path of minimal cross-sectional area and sufficient length to store the second medicament within an acceptable minimum axial package space.

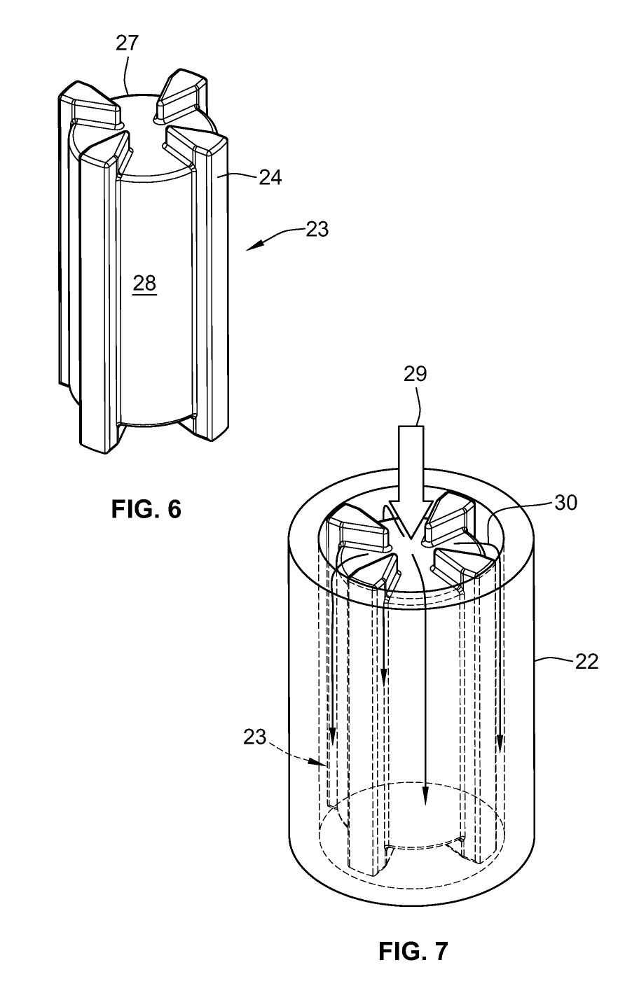

Two possible embodiments of the flow distributor 23 are illustrated in FIGS. 6-9 as cylindrical pins or inserts. Looking first to FIGS. 6 and 7, flow distributor or cylindrical insert 23 is positioned in reservoir 2 and configured such that the secondary medicament fills flow channels 27 and 28, which are defined by the shape and location of two or more support ribs 24. In a preferred embodiment, the insert 23 is located so that the edges of the flow channel 27, 28 are in direct contact with the inner walls of the reservoir 2 or the inner walls of a tube 35, when it is necessary to insert a tube 35 into the reservoir cavity 3. The flow distributor 23 (cylindrical pin) can be constructed of any material that is compatible to the primary and secondary medicaments. A preferred material is one that is typically used to manufacture septa or pistons (bungs) found in multi-dose medicament cartridges, however, any other material that is compatible with the drug could be used, e.g. glass, plastics or specific polymers. The shape of the flow channels 27, 28 can be optimized to promote plug flow of medicament, shown by arrows 29 and 30, by varying the dimensions, geometry and number of support ribs 24. The cross-sectional area of the annulus formed between the flow distributor 23 and the wall of the reservoir cavity 3 or inserted tube 35 should be kept relatively small. The volume available to store the secondary medicament would equal the internal volume of the reservoir 2 minus the volume of the flow distributor 23. Therefore, if the volume of the flow distributor 23 is marginally smaller than the internal volume of the reservoir 2, a small volume is left which the secondary medicament occupies. Hence, the scale of both the reservoir 2 and the flow distributor 23 can be large while storing a small volume of medicament. As such, the external package or reservoir geometry is not dictated by the volume of medicament. As a result, for small volumes of secondary medicament (e.g. 5-500 microliters) the package 1 can be of an acceptable size for handling, transport, manufacture and assembly. By using different pin geometries it would also be relatively easy to manufacture a range of needles 31 with different fixed microdose volumes, without changing the external dimensions of the reservoir 2, and thus, with no implications on the rest of the medicated module sub-assembly. As shown in FIG. 7, the flow as indicated by arrow 30 is initially radial from the needle 31 until the flow reaches the annulus formed between the cavity walls 22 and the flow distributor 23.

Preferably, a fluid dynamics computer simulation program, taking into account hydrodynamics and thermodynamics, would be used to obtain the optimum flow distributor design. Minimizing or eliminating reverse flow of the secondary medicament within the reservoir 2 will greatly enhance the percent expulsion from the system. Removing or reducing an adverse pressure gradient within the reservoir 2 can avoid recirculation. This can be accomplished by having a constant cross-section in the reservoir 2 to achieve an annular flow pattern. Alternatively, changing the flow direction using radial flow and/or circumferential flow (i.e. swirl) could avoid an adverse pressure gradient. As mentioned, the volume of secondary medicament can be adjusted by manipulating the volume occupied/displaced by the flow distributor 23.

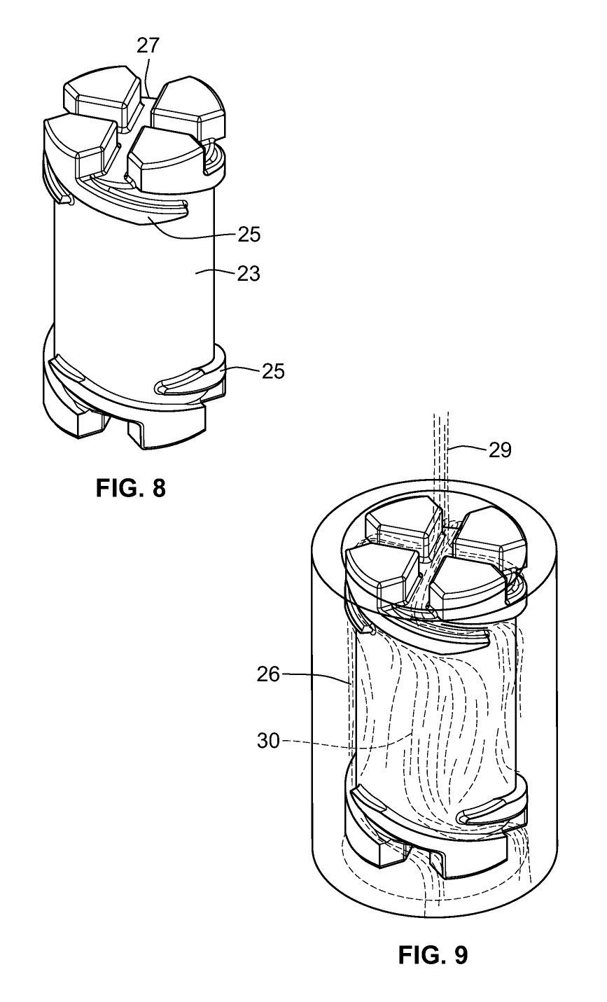

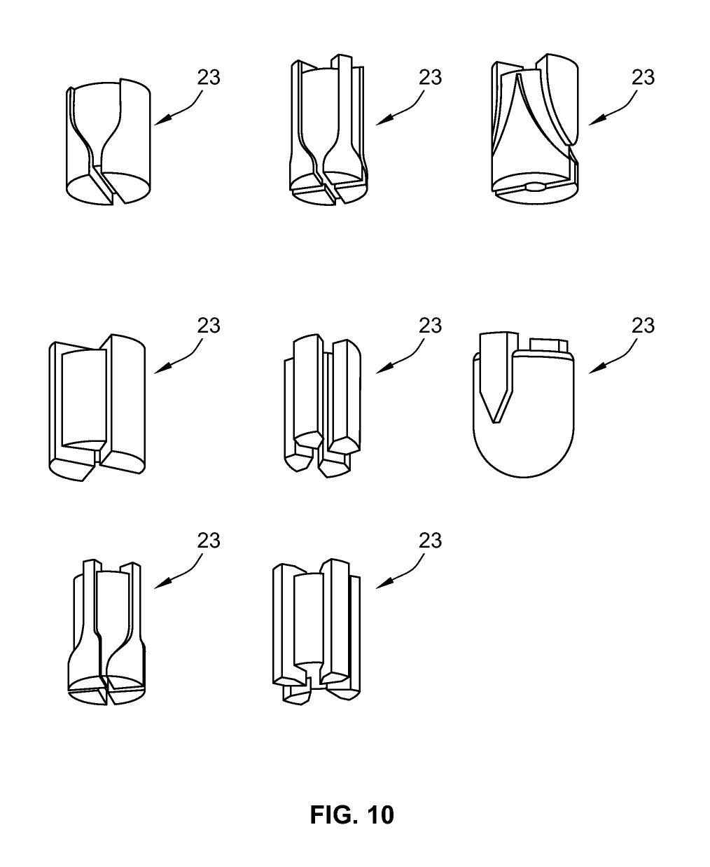

To further remove the possibility of stagnant volume at the surface of the lower sealing disc/septa 15 and to reduce the possibility of forming a vortex at the transition between the radial flow portion and the annular flow portion, FIGS. 8 and 9 show another embodiment of the flow distributor 23 that includes one or more radial vanes 25. In addition, the width of the channels 27 present in the radial flow portion are narrowed to accelerate the flow at the base to help expel the secondary medicament and to reduce the stagnant volume present on the lower septa 15. Additionally, radial features like these may also help reduce/eliminate medicament loss during the filling process due to splashing as the flow distributor 23 is inserted. In essence they would help acting like baffle plates to deflect/contain any upward splashing that occurs. FIG. 9 shows a flow pattern through the reservoir 2 obtained from computational fluid dynamic modeling. Using the flow distributor 23 shown in FIGS. 8 & 9, computer modeling predicts that less than 5% residual volume of the secondary medicament in the reservoir 2 will remain in the reservoir 2 after dose delivery, thus achieving about 95% expulsion. FIG. 10 illustrates alternate designs of the flow distributor 23 that could be used in the present disclosure. Of course, as mentioned above, the flow distribution system could equally comprise a combination of structural components within the medicated module 4. For example, the inner surface of the reservoir or vial 2 could be configured with grooves and ribs to define fluid flow channels and the seals 15, 16 could be configured to assist in changing fluid flow from axial to radial to swirl or vice versa. Likewise, various parts of the flow distribution system could be in-molded during the fabrication of the reservoir 2 and, preferably, manufactured from the same plastic material as the reservoir wall, however, other materials suitable for two- or multi-component injection molding could be used. An exemplary embodiment is shown in FIG. 11 and FIG. 12 where a flow distributor 23a is in-molded with reservoir wall.

The advantage of using in-molding techniques is that the manufacturing steps in the aseptic area are reduced to two steps only, filling (e.g. vacuum filling) and closing the primary pack with a closure/lid component. Current state of the art uses four steps of manufacturing: 1) closing the lower end of the tube; 2) filling; 3) insertion of the flow distributor; 4) closing of the upper end of the reservoir cavity. Reducing the number of manufacturing steps allows for significant improvement of equipment efficiency, process robustness, investment costs reduction, and improvement of aseptic processes.

Attachment of the medicated module 4 to the multi-use device 7 causes an engagement needle 33 located in the proximal end of module 4 to penetrate a septum sealing the distal end of cartridge 34 of the multi-use device 7. Once the engagement needle 33 has passed through the septum of the cartridge 34, fluid connection is made between the primary medicament and the needle 33. The dose of the multi-use device 7 is then set using a dose setter 12 (see FIG. 1) in the normal manner (e.g. by dialing out the appropriate number of units or cocking the device 7 if only a single dose or a fixed dose is possible). The seals 15, 16 containing the secondary medicament are pierced with needles 31 and 33 when the package 1 is moved axially relative to needle 31 (e.g. during attachment, or under the action of a needle guard or similar insertion trigger). Dispense of the medicaments is then achieved by subcutaneously injecting the medicaments via activation of a dose button 13 on device 7. The dose button 13 can be any triggering mechanism that causes the dose of the first medicament that was set by the dose setter 12 to move distally towards the distal end 32 of the device 7. In a preferred embodiment, the dose button 13 is operably connected to a spindle that engages a piston in the primary reservoir or cartridge 34 of the first medicament.

The package 1 can also include a bypass channel 50 as shown in FIG. 13 that is incorporated as part of reservoir 2 to facilitate priming of output needle 31 with the primary medicament. Alternatively, the bypass 50 can be used to directly dispense the primary medicament without the microdose of the secondary medicament. A number of designs for this bypass channel 50 can be used, for example, the needle 33 that is in fluid communication with the primary medicament could be in fluid communication with bypass channel 50 that allows the primary medicament to flow around the reservoir 2 and into a lower cavity and out through the dispense needle 31. After the optional priming operation is complete or after performing a direct dispense of the primary medicament, the package 1 can be moved axially in the proximal direction to fully engaged the upper (proximal) needle 33. Preferably, this can be performed by fully attaching the medicated module 4, rotated in the case of screw threads, to the multi-use injection device 7. This would cause both needles 31, 33 to pierce the lower and top sealing discs 15, 16 of the reservoir 2, respectively, thus opening fluid communication between the primary and microdose medicaments allowing them to be dispensed through operation of the dispense mechanism on the multi-use device 7. When this occurs the bypass channel 50 is isolated from the contents of the reservoir 34.

The connection or attachment between the medicated module 4 of the above described embodiments may contain additional features (not shown), such as connectors, stops, splines, ribs, grooves, and the like design features, that ensure that the specific medicated module 4 is attachable only to matching drug delivery device 7. Such additional features would prevent the insertion of a non-appropriate medicated module to a non-matching injection device.

The shape of the medicated module 4 may be a cylindrical body or any other geometric shape suitable for holding the reservoir 2 of the secondary medicament and for attaching one or more needle cannula 31, 33. The medicated module 4 can be manufactured from glass or other drug contact suitable material. The integrated injection needle 31 can be any needle cannula suitable for subcutaneous or intramuscular injection.

Additionally, the medicated module 4 could incorporate a safety shield device that would prevent accidental needle sticks and reduce the anxiety experienced by users who suffer from needle phobia. The exact design of the safety shield is not critical. However, a preferred design is one that is operably connected to the reservoir 2 to assist in providing the fluid communication with the primary medicament. Preferably the medicated module 4 is provided by a manufacturer as a stand-alone and separate device that is sealed to preserve sterility. The sterile seal of the module 4 is preferably designed to be opened automatically, e.g. by cutting, tearing or peeling, when the medicated module 4 is advanced or attached to the drug delivery device 7 by the user. Features such as angled surfaces on the end of the injection device 7 or features inside the module 4 may assist this opening of the seal.

The medicated module 4 should be designed to operate in conjunction with a multiple use injection device 7, preferably a pen-type multi-dose injection device, similar to what is illustrated in FIG. 1. The injection device 7 could be a reusable or disposable device. By disposable device it is meant an injection device that is obtained from the manufacturer preloaded with medicament and cannot be reloaded with new medicament after the initial medicament is exhausted. The device 7 may be a fixed dose or a settable dose device. It can also be a multi-dose device or a single use device.

A typical injection device 7 contains the previously mentioned cartridge 34 or other reservoir of medication. This cartridge 34 is typically cylindrical in shape and is usually manufactured in glass. The cartridge 34 is sealed at one end with a rubber bung and at the other end by a rubber septum. The injection pen 7 is designed to deliver multiple injections. The delivery mechanism is typically powered by a manual action of the user. However, the injection mechanism may also be powered by other means such as a spring, compressed gas or electrical energy.

Exemplary embodiments of the present invention have been described. Those skilled in the art will understand, however, that changes and modifications may be made to these embodiments without departing from the true scope and spirit of the present invention, which is defined by the claims.

1. A primary package for use in a medicated module comprising,
a. a reservoir having a proximal end, a distal end and a reservoir cavity, wherein the reservoir is configured to contain a medicament;
b. connectors located at the proximal and distal ends of the reservoir;
c. a top closure engaged with the connector located at the proximal end of the reservoir;
d. a bottom closure engaged with the connector located at the distal end of the reservoir;
e. a top seal fitted between the top closure and the proximal end of the reservoir; and
f. a bottom seal fitted between the bottom closure and the distal end of the reservoir,
wherein the connectors are configured to allow the closures to be securely attached to the reservoir such that the respective seal is maintained in a liquid tight seal with the reservoir, and wherein the connectors and closures are connected by a snap-lock engagement.

2. The primary package of claim 1, wherein the reservoir comprises a wall comprising an outer surface and an inner surface, and wherein the connectors are arranged in the outer surface of the wall.

3. The primary package of claim 1, wherein the cavity contains a microdose of the medicament, and wherein the volume of the medicament is between about 5 microliters and about 500 microliters.

4. The primary package according to claim 1, wherein the reservoir, the top and bottom closures, the connectors and/or the seals are fabricated from materials selected from the group consisting of medical grade of plastic, silicon oxide coated plastic, glass, silicon, thermoplastic elastomers, rubber elastomers and a combination thereof.

5. The primary package according to any of the previous claims, wherein the cavity contains a flow distribution system which is configured to minimize stagnant flow of the medicament out of the reservoir.

6. The primary package of claim 5, wherein the flow distribution system comprises an insert that causes radial flow of medicament, wherein the insert comprises two or more ribs or grooves defining one or more medicament flow channels, wherein the medicament fills the flow channels.

7. The primary package according to claim 5,wherein the flow distribution system comprises one or more radial vanes.

8. The primary package according to claim 5, wherein the flow distribution system is in-molded in or permanently fixed to the reservoir cavity.

9. The primary package according to claim 5, wherein the flow distribution system is attached to at least one of the top closure, the bottom closure, and at least one component of the inner surface of the wall, and wherein the size of the flow distribution system is chosen such that the cross-sectional area of an annulus formed between the flow distribution system and the wall of the cavity is minimized such that a small volume is left for storing the medicament in the cavity.

10. The primary package according to claim 5, wherein the size of the reservoir and the flow distribution system is chosen such that the package comprises a size which is suited for handling, transport, manufacture and assembly.

11. The primary package according to claim 5, wherein the volume of the flow distribution system is marginally smaller than the internal volume of the reservoir, leaving a small volume in the range of from about 5 to about 500 microliters to be occupied by the medicament.

12. The primary package according claim 1 further comprising a gasket located on the proximal end of the reservoir and being in contact with at least one of the top closure, the distal end of the reservoir or the bottom closure, wherein the gasket is configured to seal the cavity.

13. The primary package according to claim 1, wherein the reservoir has a bypass channel that connects the proximal end to the distal end and that is not in fluid communication with the cavity.

14. The primary package according to claim 3, wherein the microdose is a single microdose of the medicament, and wherein the microdose is a liquid medicament.

15. A medicated module attachable to a drug delivery device, the drug delivery device comprising a cartridge of a primary medicament and the medicated module comprising a first needle, a second needle and the primary package according to any of the previous claims, wherein the primary package is configured for fluid engagement with the first and second needles.

16. The medicated module according to claim 15, wherein the primary package is arranged in the medicated module such that the first and second needles pierce the top and bottom seals.

17. The medicated module according to claim 15, wherein the flow distribution system has proximal and distal needle cavities which are configured to accept the needles and to prevent the needles from being damaged by the flow distribution system.

18. The medicated module according to claim 15, wherein the flow distribution system is configured to prevent mixing of the medicament of the primary package with the primary medicament when the primary medicament is moved in the distal direction.

19. Method for dispensing a fixed microdose of a secondary medicament from a reservoir which is suited to minimize a volume in an inner reservoir cavity comprising the steps of:
providing the medicated module according to claim 15,
attaching the medicated module to a drug delivery device, the drug delivery device comprising a cartridge holding a primary medicament,
setting a dose of the primary medicament,
moving the set dose of the primary medicament from the cartridge in a distal direction such that the microdose of the secondary medicament is moved from the reservoir.

20. A flow distribution system configured for reducing a volume of a medicament reservoir holding a medicament, wherein the flow distribution system comprises a main body configured to ensure that the medicament is arranged on an outer part of the volume of the medicament reservoir, and wherein the flow distribution system comprises at least one rib or at least one groove defining one or more medicament flow channels, wherein the medicament fills the one or more flow channels.
